(19) United States
(12) Patent Application Publication
(10) Pub. No.: US 2024/0245780 A1
(43) Pub. Date: Jul. 25, 2024

(54) PHARMACEUTICAL COMPOSITIONS CONTAINING STABLE AMORPHOUS SOLID SOLUTIONS AND DISPERSIONS

(71) Applicants: Eastman Chemical Company, Kingsport, TN (US); The Texas A&M University System, College Station, TX (US)

(72) Inventors: Ziyaur RAHMAN, College Station, TX (US); Eman M. MOHAMED, College Station, TX (US); Sathish DHARANI, College Station, TX (US); Tahir KHUROO, College Station, TX (US); Mansoor A. KHAN, College Station, TX (US); Phillip Michael COOK, Kingsport, TN (US); Rajendran ARUNAGIRI, Hendersonville, TN (US)

(73) Assignees: Eastman Chemical Company, Kingsport, TN (US); The Texas A&M University System, College Station, TX (US)

(21) Appl. No.: 18/562,601

(22) PCT Filed: May 10, 2022
(86) PCT No.: PCT/US2022/028570

§ 371 Date: Nov. 20, 2023

Related U.S. Application Data
(60) Provisional application No. 63/192,374, filed on May 24, 2021.

(51) Int. Cl. A61K 47/26; A61K 9/20; A61K 9/48; A61K 31/436; A61K 31/437; A61K 31/496; A61K 31/506; A61K 31/5365; A61K 31/5377; A61K 31/55; A61K 38/13

(52) U.S. Cl. CPC A61K 47/26; A61K 9/2009; A61K 9/2013; A61K 9/2018; A61K 9/2054; A61K 9/2059; A61K 9/2095; A61K 9/4825; A61K 9/485; A61K 9/4858; A61K 9/4866; A61K 31/436; A61K 31/437; A61K 31/496; A61K 31/506; A61K 31/5365; A61K 31/5377; A61K 31/55; A61K 38/13

(57) ABSTRACT

Pharmaceutical compositions comprising poorly water-soluble drugs (PSDs) and sucrose acetate isobutyrate (SAIB). The compositions are amorphous solid solutions or amorphous solid dispersions where the PSDs are present in the molecular or the amorphous state in the SAIB. The PSDs in amorphous form in the amorphous solid solutions can be stable against crystallization on exposure to elevated temperature and humidity conditions. Oral dosage forms containing the compositions are characterized by a higher dissolution rate in water, a higher serum maximum concentration (Cmax), and/or a greater area under the curve (AUG) than an equivalent oral dosage form without the SAIB.

### CROSS-REFERENCE TO RELATED APPLICATION

This application claims priority to U.S. Provisional Application No. 63/192,374 filed on May 24, 2021; the entire content of which is hereby incorporated by reference.

### FIELD OF THE INVENTION

The invention generally relates to the field of pharmaceutical preparations and manufacturing. It particularly relates to pharmaceutical compositions containing amorphous solid solutions or amorphous solid dispersions, processes for making the same, and methods of increasing dissolution rate and extent, and of modulating dissolution behavior, oral absorption, and/or bioavailability of poorly water-soluble drugs.

### BACKGROUND OF THE INVENTION

### Poorly Water-Soluble Drugs

Approximately 75-90% of discovered drugs display solubility-limited absorption, which presents the pharmaceutical industry with a “poor solubility challenge”. Such drug candidates tend to display high lipophilicity, poor aqueous solubility, and consequently, low oral bioavailability.

Various pharmaceutical approaches have been proposed to improve the properties of such drugs. These approaches include salt formation, polymorphs, amorphization, complexation, particle size reduction, and drug delivery systems, e.g., liposome, microemulsion, etc.

High lattice energy, which is often reflected by a high melting temperature, is an impediment to attain adequate solubility. Any approach that reduces or eliminates lattice energy would enhance apparent solubility, such as metastable and amorphous polymorph.

In amorphization, a crystalline drug is transformed to amorphous form. Amorphous form is, by definition, a non-crystalline material which possesses no long-range order. It behaves both as a glassy solid (hard and brittle, high viscosity, low molecular mobility) and a super-cooled liquid (softer and rubbery state, low viscosity, high molecular mobility), depending on the temperature. The transition from the glassy to the rubbery state is characterized by a temperature called the glass-transition temperature (T_{g}). An increase in the molecular mobility of an amorphous material above its T_{g} is usually also accompanied by devitrification to a more stable crystalline form. As a result, a high glass-transition temperature, amorphous form of a drug is often preferred.

Amorphous drugs tend to exhibit supersaturated solubility due to their lack of crystalline order at the molecular level, and thus, tend to have a higher dissolution rate and extent followed by greater absorption. However, the amorphous form of a drug is thermodynamically unstable and may convert to the more stable crystalline form at ambient conditions, or conversion can happen at a faster rate at exaggerated temperature and humidity conditions. Due to this reason, amorphous drugs are not typically used as such in dosage forms.

To increase the thermodynamic stability of an amorphous drug, hydrophilic or hydrophobic polymers are typically added. Hydrophilic polymers are often preferred over hydrophobic ones due to their ability to increase the wettability, dispersion, and dissolution rate of the drug. Most commonly used hydrophilic polymers are hydroxypropyl methylcellulose (HPMC), hydroxy ethyl cellulose, hypromellose acetate succinate (HPMCAS), cellulose acetate phthalate (CAP), hydroxypropyl methylcellulose phthalate (HPMCP), hydroxypropyl cellulose (HPC), methyl cellulose, chitosan, carboxymethyl cellulose, ethyl cellulose, carboxymethyl ethyl cellulose, cyclodextrin and derivatives, lactose, poloxamers, polyvinylpyrrolidone (PVP), polymethacrylates (EUDRAGIT E, L, S, FS), polyvinylpyrrolidone-vinyl acetate copolymer (PVP/VA 64), polyvinyl acetate phthalate (PVAP), and polyethylene glycols (PEG) derivatives.

The increase in stability of an amorphous solid dispersion is a function of drug and polymer properties, concentration, processing method, storage condition, etc. The polymer can increase the stability of an amorphous drug by increasing its T_{g} and the distance between the drug molecules. However, forming amorphous solid dispersions generally does not increase the stability of the amorphous drug indefinitely or for all conditions. The amorphous drug may be relatively stable at ambient temperature and humidity conditions. However, excursion from ambient conditions (such as exposure to high temperature or humidity, which can happen during manufacturing, transportation, storage, or usage) can cause the amorphous drug to crystallize. That crystallization may cause the drug to fail dissolution specifications. Another reason for crystallization can be non-homogenous distribution of the amorphous drug in the solid dispersion, which could lead to formation of crystal nuclei in the region poor in polymer concentration and rich in the drug.

The stability of an amorphous drug in a polymer or excipient can be increased if it forms a homogenous solution instead of a dispersion, since solutions are thermodynamically more stable.

### Sucrose Acetate Isobutyrate

Sucrose acetate isobutyrate (SAIB) was introduced as a food additive in 1969 and is approved as a food additive for use in carbonated and non-carbonated beverages in over 28 countries. Although SAIB has found many uses in the coatings industry, its only use in foods at the present time is as a “weighting” or “density-adjusting” agent in citrus-based carbonated or non-carbonated beverages. That is, SAIB has the ability, when mixed with flavor oils (e.g., orange oil), to adjust the density of the oil-weighting agent blend to stabilize the emulsion of the flavor oil in water. The maximum level of SAIB permitted in soft drinks in any country is 500 ppm. Extensive safety data on SAIB are available, both in animals (mice, rats, and dogs) and in humans, including children.

SAIB is classified as “Generally Recognized as Safe” (GRAS) by the USFDA and as a food additive by the European Union as indicated by “E” in the number E444.

In 1996, the Joint FAO/WHO Expert Committee on Food Additives (JECFA) assigned a permanent acceptable daily intake for SAIB of up to 20 mg/kg body weight including in pediatric populations.

SAIB has been disclosed in depot formulations (see, e.g., U.S. Pat. No. 10,682,340) as well as in oral dosage forms (see, e.g., U.S. Pat. No. 8,133,507).

There is a continuing need to provide alternative and/or improved methods for increasing the solubility, dissolution, and/or oral bioavailability of poorly water-soluble drugs.

The present invention addresses this need as well as others, which will become apparent from the following description and the appended claims.

### SUMMARY OF THE INVENTION

The invention is as set forth in the appended claims.

Briefly, in one aspect, the invention provides a pharmaceutical composition. The composition comprises:
(a) a poorly water-soluble drug (PSD);
(b) sucrose acetate isobutyrate (SAIB);
(c) less than 0.01 wt % of a solvent, based on the total weight of the composition; and
(d) optionally, at least one pharmaceutically acceptable excipient which is insoluble in the SAIB,
wherein the composition:
   (i) is an amorphous solid solution or an amorphous solid dispersion;
   (ii) has a mass ratio of PSD to SAIB ranging from 0.1:1 to 1:5; and
   (iii) has a mass ratio of excipient to SAIB ranging from 0:1 to 100:1, and wherein the PSD is present in the molecular or amorphous state in the SAIB.

In another aspect, the invention provides an oral dosage form comprising the pharmaceutical composition of the invention.

In yet another aspect, the invention provides processes for preparing the pharmaceutical compositions of the invention. In one iteration, the process comprises:
heating the SAIB to a temperature sufficient to dissolve the PSD and for the PSD to exist in the molecular or amorphous state in the SAIB;
adding the PSD and optionally, the at least one pharmaceutically acceptable excipient, to the heated SAIB with mixing to dissolve the PSD and to form a mixture; and
cooling the mixture to form a pharmaceutical composition comprising an amorphous solid solution or an amorphous solid dispersion.

In another iteration, the process comprises:
forming a mixture comprising the PSD, the SAIB, an organic solvent, and optionally, the at least one pharmaceutically acceptable excipient;
granulating the mixture to form granules; and
removing the organic solvent from the granules to form a pharmaceutical composition comprising an amorphous solid solution or an amorphous solid dispersion.

### BRIEF DESCRIPTION OF THE DRAWINGS

FIG. 1 shows photographs of physical mixtures as well as amorphous solid solutions of various drugs before and after exposure to 40° C./75% RH from Example 2.

FIG. 2 shows NIR spectra of (A) SAIB, aprepitant, aprepitant-SAIB physical mixture, and aprepitant AmSSol before and after exposure to 40° C./75% RH; (B) SAIB, aripiprazole, aripiprazole-SAIB physical mixture, and aripiprazole AmSSol before and after exposure to 40° C./75% RH; (C) SAIB, carbamazepine, carbamazepine-SAIB physical mixture, and carbamazepine AmSSol before and after exposure to 40° C./75% RH; and (D) SAIB, cyclosporine, cyclosporine-SAIB physical mixture, and cyclosporine AmSSol before and after exposure to 40° C./75% RH.

FIG. 3 shows NIR spectra of (A) SAIB, dolutegravir, dolutegravir-SAIB physical mixture, and dolutegravir AmSSol before and after exposure to 40° C./75% RH; (B) SAIB, rifaximin, rifaximin-SAIB physical mixture, and rifaximin AmSSol before and after exposure to 40° C./75% RH; (C) SAIB, sirolimus, sirolimus-SAIB physical mixture, and sirolimus AmSSol before and after exposure to 40° C./75% RH; and D) SAIB, tacrolimus, tacrolimus SAIB physical mixture, and tacrolimus AmSSol before and after exposure to 40° C./75% RH.

FIG. 4 shows NIR hyperspectroscopy images of a physical mixture of drug and SAIB, and the corresponding AmSSol initially and after exposure to 40° C./75% RH.

FIG. 5 shows X-ray powder diffractograms of (A) aprepitant, aprepitant-SAIB physical mixture, and aprepitant AmSSol before and after exposure to 40° C./75% RH; (B) aripiprazole, aripiprazole-SAIB physical mixture, and aripiprazole AmSSol before and after exposure to 40° C./75% RH; (C) carbamazepine, carbamazepine-SAIB physical mixture, and carbamazepine AmSSol before and after exposure to 40° C./75% RH; and (D) cyclosporine, cyclosporine-SAIB physical mixture, and cyclosporine AmSSol before and after exposure to 40° C./75% RH.

FIG. 6 shows NIR spectra of (A) dolutegravir, dolutegravir-SAIB physical mixture, and dolutegravir AmSSol before and after exposure to 40° C./75% RH; (B) rifaximin, rifaximin-SAIB physical mixture, and rifaximin AmSSol before and after exposure to 40° C./75% RH; (C) sirolimus, sirolimus-SAIB physical mixture, and sirolimus AmSSol before and after exposure to 40° C./75% RH; and (D) tacrolimus, tacrolimus-SAIB physical mixture, and tacrolimus AmSSol before and after exposure to 40° C./75% RH.

FIG. 7 is a graph of the dissolution curves of the aprepitant formulations in Example 4.

FIG. 8 is a graph of the dissolution curves of an aprepitant formulation before and after exposure to 40° C./75% RH for 1 month and 3 months from Example 5.

FIG. 9 is a graph of the dissolution curves of an aprepitant formulation before and after exposure to 25° C./60% for 3 months from Example 6.

FIG. 10 is a graph of the dissolution curves of the tacrolimus formulations in Example 9.

FIG. 11 is a graph of the dissolution curves of a tacrolimus formulation before and after exposure to 40° C./75% RH for 1 month and 2 months from Example 10.

FIG. 12 is a graph of the dissolution curves of a tacrolimus formulation before and after exposure to 25° C./60% RH for 3 months from Example 11.

### DETAILED DESCRIPTION OF THE INVENTION

It has been surprisingly discovered that poorly water-soluble drugs (PSDs) can be formulated with sucrose acetate isobutyrate (SAIB) to form stable amorphous solid solutions or amorphous solid dispersions. The PSDs are present in the molecular or the amorphous state in the SAIB. The PSDs in amorphous form in the amorphous solid solutions can be stable against crystallization on exposure to elevated temperature and humidity conditions. Oral dosage forms containing the amorphous solid solutions or amorphous solid dispersions can have higher water dissolution rates, higher serum maximum concentrations (Cₘₐₓ), and/or greater areas under the curve (AUC) than equivalent oral dosage forms without the SAIB.

### Definitions

To facilitate understanding the invention, several terms are defined below. Those left undefined have meanings as commonly understood by a person of ordinary skill in the technical areas relevant to the present invention.

The abbreviation “SAIB” refers to sucrose acetate isobutyrate. SAIB has CAS Registry No. 27216-37-1. SAIB may be obtained commercially from Eastman Chemical Company, Kingsport, Tennessee.

The terms “poorly water-soluble drugs,” “poorly soluble drugs,” and “PSDs” refer to drugs having a solubility such that the dose to be administered cannot be dissolved in 250 ml of aqueous media within the pH range of 1 to 6.8 at 37° C. and atmospheric pressure. Examples of such drugs include tacrolimus and aprepitant.

The term “crystalline” describes a solid having a highly regular chemical structure in the crystalline lattice. Crystalline solids have a sharp melting point and characteristic sharp peaks in X-ray powder diffractogram.

The term “amorphous” describes a solid material having no long-range order in the position of its atoms. Amorphous solids are generally supercooled liquids in which the molecules are arranged in a random manner so that there is no well-defined arrangement and no long-range order. Amorphous solids are generally isotropic, i.e., they exhibit similar properties in all directions and do not have definite melting points. For example, an amorphous material is a solid material having no sharp characteristic crystalline peak(s) in its X-ray powder diffraction (XRPD) pattern. Instead, one or several broad peaks (e.g., halos) appear in its XRPD pattern. Broad peaks are characteristic of an amorphous solid.

In both amorphous solid solutions (AmSSols) and amorphous solid dispersions (AmSDs), the term “amorphous” characterizes the state of the PSD molecules. The PSD molecules exist in the high-energy, amorphous state. The amorphous PSD molecules are stabilized within the solid SAIB matrix to prevent recrystallization.

By “molecular state”, it is meant the PSD exists as a molecular dispersion in the SAIB, or stated another way, the PSD is molecularly dispersed in the SAIB.

By “amorphous state”, it is meant the PSD exists in an amorphous form dispersed in the SAIB.

A “solid solution”, like its liquid counterpart, has only one phase, regardless of the number of components, and that phase is solid at room temperature and pressure. In true solid solutions, one of the solid components is completely dissolved in the other solid component.

A “physical mixture” describes a mixture where the components are simply mixed. At most, there is incomplete phase transformation of the drug into solution or amorphous form. The phase transformation may be confirmed by various analytical tools, such as powder X-ray diffraction, DSC, and spectroscopic method. Typically, at least some of the drug in a physical mixture is in crystalline form.

The terms “organic solvent” and “solvent” refer to a pharmaceutically acceptable solvent which can aid the drug to solubilize in the SAIB. The pharmaceutically acceptable solvent may be selected from Class 2 or Class 3 solvents per the International Council for Harmonization of Technical Requirements for Pharmaceuticals for Human Use (ICH) Q3C Impurities: Guideline for Residual Solvents. Examples of pharmaceutically acceptable solvents include ethanol, isopropanol, 1-butanol, acetic acid, etc.

The terms “stable” and “stability” mean that an amorphous drug in an amorphous solid solution or an amorphous solid dispersion does not change into one or more crystalline physical forms (e.g., different solid forms as measured by XRPD, DSC, etc.) or does not produce a significant fraction (e.g., ≤25 wt %) of the crystalline drug when subjected to specified conditions, e.g., room temperature and ambient humidity or 40° C. at 75% relative humidity, for a specified period of time, e.g., 1 day, 2 days, 3 days, 1 week, 2 weeks, 1 month, 2 months, 3 months, 6 months, 12 months, 18 months, 24 months, or longer.

The terms “pharmaceutically acceptable excipient,” “excipient,” and “pharmaceutically acceptable carrier” are used interchangeably. They refer a substance, other than the PSD and the SAIB, with which the drug is formulated. The excipient generally does not provide any pharmacological activity to the formulation, though it may provide chemical and/or biological stability, and/or release characteristics. The pharmaceutically acceptable excipient can be selected from the USFDA's “Inactive Ingredient Database” or those falling within the “Generally Recognized As Safe” (GRAS) category.

Categories of excipients include diluents, disintegrants, super-disintegrants, lubricants, glidants, binders, hydrophilic polymers, surfactants, coatings, etc. Examples of diluents include lactose, mannitol, dibasic calcium phosphate, tribasic calcium phosphate, isomalt, etc. Examples of disintegrants and super-disintegrants include sodium starch glycolate, sodium croscarmellose, microcrystalline cellulose, gelatinized starch, mannitol, etc. Examples of lubricants include magnesium stearate, stearic acid, sodium stearyl fumarate, etc. Examples of binders include HPMC, HPC, chitosan, poloxamers, PVP, etc. Examples of surfactants include sodium lauryl sulfate, poloxamers, etc.

The term “insoluble” means that the substance's solubility is less than 1 g per 10,000 mL of solvent at room temperature and pressure.

The term “immediate release” or “immediate-release” means a release of the drug to an environment over a period of seconds to no more than about 120 minutes.

The term “extended release” or “extended-release” assumes the definition as widely recognized in the art of pharmaceutical sciences. An extended-release dosage form will release the drug at a substantially constant rate over an extended period (e.g., 4, 6, 8, 10, 12, or 24 hours) or a substantially constant amount of the drug will be released incrementally over an extended period (e.g., 4, 6, 8, 10, 12, or 24 hours).

The term “oral bioavailability” refers to the degree to which a drug becomes available to the systemic circulation after being absorbed from the gastrointestinal tract. Poor oral bioavailability is a significant problem encountered in the development of pharmaceutical compositions, particularly those containing an active ingredient that is poorly water soluble.

The term “total number of donor hydrogen” refers to the total number of nitrogen-hydrogen and oxygen-hydrogen bonds in a molecule.

### Pharmaceutical Compositions

In one aspect, the invention provides a pharmaceutical composition. The composition comprises:
(a) a poorly water-soluble drug (PSD);
(b) sucrose acetate isobutyrate (SAIB);
(c) less than 0.01 wt % of a solvent, based on the total weight of the composition; and
(d) optionally, at least one pharmaceutically acceptable excipient which is insoluble in the SAIB,
wherein the composition:
   (i) is an amorphous solid solution or an amorphous solid dispersion;
   (ii) has a mass ratio of PSD to SAIB ranging from 0.1:1 to 1:5; and
   (iii) has a mass ratio of excipient to SAIB ranging from 0:1 to 100:1, and
wherein the PSD is present in the molecular or amorphous state in the SAIB.

The PSD may be selected from either Class II or Class IV of the Biopharmaceutical Classification System (BCS). Both classes are known for their low solubility in water. Examples of such drugs include aprepitant, aripiprazole, carbamazepine, cyclosporine, dolutegravir, rifaximin, sirolimus, and tacrolimus.

The PSD may have a solubility in the SAIB of at least 0.1 mg/g, at least 0.4 mg/g, at least 1.0 mg/g, at least 1.5 mg/g, at least 2.0 mg/g, at least 10 mg/g, at least 20 mg/g, at least 30 mg/g, at least 50 mg/g, at least 75 mg/g, at least 100 mg/g, at least 150 mg/g, or at least 200 mg/g, at 130° C. or 150° C. and atmospheric pressure.

In various embodiments, the PSD can have a molecular mass of at least 500 g/mol, at least 750 g/mol, at least 1000 g/mol, or at least 1200 g/mol. Surprisingly, it has been discovered that the molecular mass of the PSD can have a positive effect on its solubility in the SAIB. As the molecular mass of the PSD increases, its solubility in the SAIB can also increase.

In various embodiments, the PSD can have a total number of donor hydrogen (TNDH) of at least 1, at least 2, at least 3, at least 4, or at least 5 per molecule. Surprisingly, it has been discovered that the TNDH in a PSD molecule can have a positive effect on its solubility in the SAIB. As the TNDH increases, the solubility of the PSD in the SAIB can also increase.

In various embodiments, the PSD can have a melting point of 250° C. or less, 225° C. or less, 200° C. or less, 195° C. or less, 190° C. or less, 185° C. or less, 180° C. or less, 175° C. or less, 170° C. or less, 165° C. or less, 160° C. or less, 155° C. or less, or 150° C. or less. Alternatively, or additionally, the PSD can have a melting point of at least 140° C., at least 145° C., at least 150° C., at least 155° C., at least 160° C., at least 165° C., at least 170° C., at least 175° C., at least 180° C., at least 185° C., at least 190° C., at least 195° C., at least 200° C., at least 205° C., at least 210° C., at least 215° C., at least 220° C., at least 225° C., at least 230° C., at least 235° C., at least 240° C., or at least 245° C. Surprisingly, it has been discovered that the melting point of the PSD can have a negative effect on its solubility in the SAIB. Generally, the higher the melting point of the PSD, the lower its solubility can be in the SAIB when solubilized at the same temperature.

In various embodiments, the PSD can have a partition coefficient (log P) of 2.6 to 3.3. Surprisingly, it has been discovered that too high or too low of a log P can have a negative effect on the PSD's solubility in the SAIB. For example, PSDs that have a log P below 2.6 or above 3.3 tend to have very little or negligible solubility in the SAIB.

The PSD may advantageously have any combination of two or more of the solubility, molecular mass, total number of hydrogen donor, melting point, and log P parameters described herein.

The mass ratio of PSD to SAIB in the pharmaceutical composition can also range from 0.1:1 to 1:4, from 0.1:1 to 1:3, from 0.1:1 to 1:2, or from 0.1:1 to 1:1.

In various embodiments, the pharmaceutical composition comprises 0.001 wt % or less of solvent, based on the total weight of the pharmaceutical composition.

In various embodiments, the pharmaceutical composition is free of solvent.

In various embodiments, the pharmaceutical composition is free of cellulose ester.

In various embodiments, the pharmaceutical composition is free of cellulose acetate butyrate.

In various embodiments, the pharmaceutical composition is stable when stored in an open container at 40° C. and 75% relative humidity for at least one week.

In various embodiments, the pharmaceutical composition is stable when stored in a sealed container at room temperature and ambient humidity for at least 6 months, at least 12 months, at least 18 months, or at least 24 months.

In various embodiments, the pharmaceutical composition of the invention may have a higher dissolution rate in water than an equivalent pharmaceutical composition without the SAIB.

In various embodiments, the pharmaceutical composition of the invention may have a higher dissolution rate in water than an equivalent pharmaceutical composition comprising a physical mixture of the same ingredients.

In various embodiments, the pharmaceutical composition does not comprise the at least one excipient.

In various embodiments, the pharmaceutical composition is or comprises an amorphous solid solution (AmSSol).

In various AmSSol embodiments, the pharmaceutical composition has an extended-release profile.

In various AmSSol embodiments, the composition comprises:
(a) a poorly water-soluble drug (PSD);
(b) sucrose acetate isobutyrate (SAIB); and
(c) less than 0.01 wt % of a solvent, based on the total weight of the composition;
wherein the composition:
   (i) is an amorphous solid solution; and
   (ii) has a mass ratio of PSD to SAIB ranging from 0.1:1 to 1:5, and
wherein the PSD is present in the molecular or amorphous state in the SAIB.

In various AmSSol embodiments, the pharmaceutical composition comprises 0.001 wt % or less of solvent, based on the total weight of the pharmaceutical composition.

In various AmSSol embodiments, the pharmaceutical composition is free of solvent.

In various AmSSol embodiments, the pharmaceutical composition is free of cellulose ester.

In various AmSSol embodiments, the pharmaceutical composition is free of cellulose acetate butyrate.

An excipient may be added to the AmSSol to make an amorphous solid dispersion (AmSD). Excipients can alter the rate of water penetration into the amorphous hydrophobic drug contained in the solid dispersion, thereby changing the dissolution behavior of the formulation.

It may be desirable to incorporate more than one excipient from the same category or excipients from two or more different categories to achieve a specific dissolution profile. For example, lactose and mannitol belong to the same category of diluents, but they may be used together. Sodium lauryl sulfate and microcrystalline cellulose, on the other hand, belong to the different categories of surfactant and diluent, respectively, but they may also be used together. Microcrystalline cellulose, croscarmellose, and sodium lauryl sulfate belong to three different classes of excipients, but they may also be used together.

The excipient is desirably insoluble in the SAIB and is present in disperse form.

The excipient is also desirably insoluble in the organic solvent(s), if used.

Thus, in various AmSD embodiments, the composition comprises:
(a) a poorly water-soluble drug (PSD);
(b) sucrose acetate isobutyrate (SAIB);
(c) less than 0.01 wt % of a solvent, based on the total weight of the composition; and
(d) at least one pharmaceutically acceptable excipient which is insoluble in the SAIB,
wherein the composition:
   (i) is an amorphous solid dispersion;
   (ii) has a mass ratio of PSD to SAIB ranging from 0.1:1 to 1:5; and
   (iii) has a mass ratio of excipient to SAIB ranging from 0.01:1 to 100:1, and
wherein the PSD is present in the molecular or amorphous state in the SAIB.

The pharmaceutical composition in the form of an AmSD can have an immediate-release profile or an extended-release profile.

Dissolution of the PSD can be modulated by the relative proportion of excipients to SAIB. Generally, higher proportions of the excipient(s) favor faster penetration of water and subsequent diffusion of the PSD into the bulk medium—hence, producing an immediate dissolution profile.

For an immediate-release profile, the mass ratio of excipient(s) to SAIB can range from 5:1 to 100:1, from 10:1 to 100:1, from 20:1 to 100:1, from 30:1 to 100:1, from 40:1 to 100:1, from 50:1 to 100:1, from 60:1 to 100:1, from 70:1 to 100:1, from 80:1 to 100:1, or from 90:1 to 100:1.

On the other hand, for an extended dissolution of the drug, the mass ratio of excipient(s) to SAIB can range from 0:1 to 4:1, from 0:1 to 3:1, from 0:1 to 2:1, from 0:1 to 1:1, from 0.01:1 to 4:1, from 0.01:1 to 3:1, from 0.01:1 to 2:1, from 0.01:1 to 1:1, from 0.1:1 to 4:1, from 0.1:1 to 3:1, from 0.1:1 to 2:1, or from 0.1:1 to 1:1.

In various AmSD embodiments, the pharmaceutical composition comprises 0.001 wt % or less of solvent, based on the total weight of the pharmaceutical composition.

In various AmSD embodiments, the pharmaceutical composition is free of solvent.

In various AmSD embodiments, the pharmaceutical composition is free of cellulose ester.

In various AmSD embodiments, the pharmaceutical composition is free of cellulose acetate butyrate.

In various AmSD embodiments, the pharmaceutical composition can increase the dissolution rate and extent, and the bioavailability of the PSD compared to the PSD alone or to a physical mixture of the PSD with excipient(s). An increase in oral bioavailability is indicated by area under the plasma concentration curve and maximum plasma concentration.

Amorphous solid dispersions according to the invention can show similar physical stability as that of amorphous solid dispersions based on a hydrophilic polymer, as indicated by XRPD and dissolution.

### Processes for Preparation

The pharmaceutical compositions of the invention may be prepared by various techniques, including high-shear granulation and hot-melt extrusion.

Thus, in another aspect, the invention provides processes for preparing the pharmaceutical compositions.

One such process comprises:
heating the SAIB to a temperature sufficient to dissolve the PSD and for the PSD to exist in the molecular or amorphous state in the SAIB;
adding the PSD and optionally, the at least one pharmaceutically acceptable excipient, to the heated SAIB with mixing to dissolve the PSD and to form a mixture; and
cooling the mixture to form a pharmaceutical composition comprising an amorphous solid solution or an amorphous solid dispersion.

In order to facilitate dissolution of the PSD and for the PSD to exist in the molecular or amorphous state in the SAIB upon mixing, the SAIB may be advantageously heated to an elevated temperature. The temperature to which the SAIB should be heated depends upon the PSD to be dissolved. Some PSDs may benefit from higher temperatures than others to dissolve and exist in the molecular or amorphous state in the SAIB. The precise temperature for a particular PSD may be determined by routine experimentation.

Generally, the SAIB may be heated to a temperature, such as from 60 to 200° C., from 70 to 200° C., from 80 to 200° C., from 90 to 200° C., from 100 to 200° C., from 110 to 200° C., from 120 to 200° C., from 130 to 200° C., from 140 to 200° C., from 150 to 200° C., from 80 to 175° C., from 90 to 175° C., from 100 to 175° C., from 110 to 175° C., from 120 to 175° C., from 130 to 175° C., from 140 to 175° C., from 150 to 175° C., from 80 to 160° C., from 90 to 160° C., from 100 to 160° C., from 110 to 160° C., from 120 to 160° C., from 130 to 160° C., from 140 to 160° C., from 150 to 160° C., from 80 to 150° C., from 90 to 150° C., from 100 to 150° C., from 110 to 150° C., from 120 to 150° C., from 130 to 150° C., or from 140 to 150° C.

In many cases, it may be sufficient to heat the SAIB to a temperature of 80 to 175° C., 90 to 175° C., 100 to 175° C., 110 to 175° C., 120 to 175° C., 130 to 175° C., 80 to 160° C., 90 to 160° C., 100 to 160° C., 110 to 160° C., 120 to 160° C., 130 to 160° C., 80 to 150° C., 90 to 150° C., 100 to 150° C., 110 to 150° C., 120 to 150° C., or 130 to 150° C.

The manner of adding the PSD to the heated SAIB is not particularly limiting. The PSD may be added all at once or incrementally.

The mixture of the PSD and the heated SAIB may be mixed or blended using any conventional apparatus, such as a high-shear mixer, until the PSD is dissolved in the SAIB. The dissolution of the PSD may be confirmed by various techniques including visually and/or analytically.

The cooling step may be performed by terminating or removing the heat supply to the SAIB and/or exposing the mixture to any suitable cooling medium or apparatus.

In various embodiments, the process is carried out in the absence of an organic solvent.

In various embodiments, the process is carried out in the absence of cellulose ester.

In various embodiments, the process is carried out in the absence of cellulose acetate butyrate.

In various embodiments, one or more steps of the process may be advantageously carried out in a hot-melt extruder. For example, the heating and/or mixing steps may be conducted in an extruder, such as a twin-screw extruder. Following extrusion, the PSD/SAIB/excipient(s) mixture may be shaped and cooled or cooled and shaped as tablets, granules, pellets, sheets, strands, sticks, or powder. These intermediate forms may be further processed using conventional techniques into oral dosage forms such as tablets and capsules.

Another process for preparing the pharmaceutical compositions described herein comprises:
forming a mixture comprising the PSD, the SAIB, an organic solvent, and optionally, the at least one pharmaceutically acceptable excipient;
granulating the mixture to form granules; and
removing the organic solvent from the granules to form a pharmaceutical composition comprising an amorphous solid solution or an amorphous solid dispersion.

The organic solvent is desirably miscible with SAIB and can aid in solubilizing the PSD and/or in dispersing the other components of the formulation in the SAIB. A single organic solvent or a mixture of organic solvents may be used. Useful amounts of the organic solvent, relative to the SAIB, include a mass ratio of 0.1:1 to 1:1.

The forming and granulating steps may be performed in any suitable mixing device, such as a blender or a high-shear granulator.

The organic solvent may be removed by using techniques such as heating (e.g., at 25 to 65° C.), vacuum-drying, or both. Other solvent removal techniques include freeze-drying or spray-drying. It is desirable to remove as much solvent as possible since some solvents may not be well tolerated by some patients. The dried granules may be further processed using conventional techniques into oral dosage forms such as tablets and capsules.

### Oral Dosage Forms

In yet another aspect, the invention provides an oral dosage form comprising the pharmaceutical composition of the invention.

The oral dosage form may be a tablet or a capsule. In various embodiments, the oral dosage form is a tablet.

The oral dosage form may contain the PSD in an amount ranging from 0.5 to 500 mg, 0.5 to 450 mg, 0.5 to 400 mg, 0.5 to 350 mg, 0.5 to 300 mg, 0.5 to 250 mg, 0.5 to 200 mg, 0.5 to 150 mg, 0.5 to 100 mg, or 0.5 to 50 mg.

In various embodiments, the oral dosage form comprises 0.01 wt % or less of solvent, based on the total weight of the oral dosage form.

In various embodiments, the oral dosage form comprises 0.001 wt % or less of solvent, based on the total weight of the oral dosage form.

In various embodiments, the oral dosage form is free of solvent.

In various embodiments, the oral dosage form is free of cellulose ester.

In various embodiments, the oral dosage form is free of cellulose acetate butyrate.

In various embodiments, the oral dosage form of the invention may have a higher dissolution rate in water than an equivalent oral dosage form without the SAIB.

In various embodiments, the oral dosage form of the invention may have a higher dissolution rate in water than an equivalent oral dosage form comprising a physical mixture of the same ingredients.

In various embodiments, the oral dosage form of the invention may have a higher serum maximum concentration (Cₘₐₓ) than an equivalent oral dosage form without the SAIB.

In various embodiments, the oral dosage form of the invention may have a higher serum maximum concentration (Cₘₐₓ) than an equivalent oral dosage form comprising a physical mixture of the same ingredients.

In various embodiments, the oral dosage form of the invention may have a Cₘₐₓ of at least 1.1×, at least 1.2×, at least 1.3×, at least 1.4×, at least 1.5×, at least 1.6×, at least 1.7×, at least 1.8×, at least 1.9×, or at least 2× greater than the Cₘₐₓ of an equivalent oral dosage form without the SAIB.

In various embodiments, the oral dosage form of the invention may have a Cₘₐₓ of at least 1.1×, at least 1.2×, at least 1.3×, at least 1.4×, at least 1.5×, at least 1.6×, at least 1.7×, at least 1.8×, at least 1.9×, or at least 2× greater than the Cₘₐₓ of an equivalent oral dosage form comprising a physical mixture of the same ingredients.

In various embodiments, the oral dosage form of the invention may have a greater area under the curve (AUC) than an equivalent oral dosage form without the SAIB.

In various embodiments, the oral dosage form of the invention may have a greater area under the curve (AUC) than an equivalent oral dosage form comprising a physical mixture of the same ingredients.

In various embodiments, the oral dosage form of the invention may have an AUC of at least at least 1.1×, at least 1.2×, at least 1.3×, at least 1.4×, at least 1.5×, at least 1.6×, at least 1.7×, at least 1.8×, at least 1.9×, at least 2×, at least 2.1×, at least 2.2×, at least 2.3×, at least 2.4×, or at least 2.5× greater than the AUC of an equivalent oral dosage form without the SAIB.

In various embodiments, the oral dosage form of the invention may have an AUC of at least at least 1.1×, at least 1.2×, at least 1.3×, at least 1.4×, at least 1.5×, at least 1.6×, at least 1.7×, at least 1.8×, at least 1.9×, at least 2×, at least 2.1×, at least 2.2×, at least 2.3×, at least 2.4×, or at least 2.5× greater than the AUC of an equivalent oral dosage form comprising a physical mixture of the same ingredients.

### General Provisions

To remove any doubt, the present invention includes and expressly contemplates and discloses any and all combinations of embodiments, features, characteristics, parameters, and/or ranges mentioned herein. That is, the subject matter of the present invention may be defined by any combination of embodiments, features, characteristics, parameters, and/or ranges mentioned herein.

It is contemplated that any ingredient, component, or step that is not specifically named or identified as part of the present invention may be explicitly excluded.

Any process/method, apparatus, compound, composition, embodiment, or component of the present invention may be modified by the transitional terms “comprising,” “consisting essentially of,” or “consisting of,” or variations of those terms.

As used herein, the indefinite articles “a” and “an” mean one or more, unless the context clearly suggests otherwise. Similarly, the singular form of nouns includes their plural form, and vice versa, unless the context clearly suggests otherwise.

While attempts have been made to be precise, the numerical values and ranges described herein may be considered approximations. These values and ranges may vary from their stated numbers depending upon the desired properties sought to be obtained by the present disclosure as well as the variations resulting from the standard deviation found in the measuring techniques. Moreover, the ranges described herein are intended and specifically contemplated to include all sub-ranges and values within the stated ranges. For example, a range of 50 to 100 is intended to include all values within the range including sub-ranges such as 60 to 90, 70 to 80, etc.

Any two numbers of the same property or parameter reported in the working examples may define a range. Those numbers may be rounded off to the nearest thousandth, hundredth, tenth, whole number, ten, hundred, or thousand to define the range.

The content of all documents cited herein, including patents as well as non-patent literature, is hereby incorporated by reference in their entirety. To the extent that any incorporated subject matter contradicts with any disclosure herein, the disclosure herein shall take precedence over the incorporated content.

This invention can be further illustrated by the following working examples, although these examples are included merely for purposes of illustration and are not intended to limit the scope of the invention.

### EXAMPLES

### Analytical Methods

### HPLC

Analysis of drugs was performed by HPLC. The HPLC equipment used was Infinity 1260 (Agilent Technologies, CA, USA) fitted with a quaternary pump, an autosampler, and a UV detector, and a column compartment maintained at 30° C. The method was developed and validated by ICH 2005. Details of the HPLC method are shown in Table 1. Solubility was determined in three replicates for each drug.

TABLE 1
HPLC methods for drug analysis.

| | | | Mobile | Injection | UV Wave- | Calib. |
|---|---|---|---|---|---|---|
| | | | Flow | Volume | length | Range |
| Drug | Column | Mobile phase | Rate | (μL) | (nm) | (μg/mL) |
| Rifaximin | C8-250 × 4.6 | ACN:Water-70:30 | 1.0 | 20 | 230 | 1-50 |
| | 5 μm | | | | | |
| Dasatinib | C18-150 × 4.6 | ACN:THF:Phosphate | 1.0 | 20 | 323 | 2-50 |
| | 5 μm | buffer (pH 5.5, 10 mM)- | | | | |
| | | 20:5:75 | | | | |
| Aripiprazole | C8-100 × 4.6 | Citrate buffer (pH | 1.0 | 20 | 260 | 10-100 |
| | 5 μm | 3, 10 mM):ACN-60:40 | | | | |
| Dolutegravir | C18-Luna- | ACN:Water-60:40 | 1.0 | 10 | 230 | 10-150 |
| | 250 × 4.6 | | | | | |
| | 5 μm | | | | | |
| Cyclosporin | C8-Luna- | ACN:Water-75:25 | 1.0 | 20 | 210 | 1-10 |
| | 150 × 4.6 | | | | | |
| | 3 μm | | | | | |
| Itraconazole | C18-Luna- | ACN:Water-90:10 | 1.0 | 20 | 263 | 10-100 |
| | 250 × 4.6 | | | | | |
| | 5 μm | | | | | |
| Tacrolimus | C18-Luna- | Phosphate buffer (pH | 1.2 | 20 | 213 | 10-100 |
| | 150 × 4.6 | 3, 10 mM):ACN-60:40 | | | | |
| | 3 μm | | | | | |
| Sirolimus | C18-Luna- | Phosphate buffer (pH | 1.5 | 20 | 278 | 10-100 |
| | 150 × 4.6 | 3, 10 mM):ACN-60:40 | | | | |
| | 3 μm | | | | | |
| Aprepitant | C18-Luna- | Methanol:Phosphate | 1.0 | 20 | 210 | 1-50 |
| | 150 × 4.6 | buffer (pH 3, 10 mM)- | | | | |
| | 3 μm | 80:20 | | | | |
| Carbamazepine | C18-Luna- | ACN:Water:Methanol- | 1.0 | 20 | 254 | 10-250 |
| | 250 × 4.6 | 30:40:30 | | | | |
| | 5 μm | | | | | |

### Near-Infrared Spectroscopy (NIR)

NIR spectra of samples were obtained using modular Nicolet™ iS™ 50 system (Thermo Fisher Scientific, Austin, TX). The instrument was fitted with a scanning grating monochromator and a diffuse reflectance apparatus (rapid content analyzer). After the instrument passed the diagnostic tests and reflectance standardization, samples in 20 mL borosilicate glass vials were placed on the sample window and centered with an iris. NIR spectra ranging from 4000-10000 cm⁻¹ with a data resolution of 4 cm⁻¹ and 32 scans were obtained in sextet from the base of the vial, which was transparent to the NIR.

### Near-Infrared Hyperspectroscopy

Via-Spec II Hyperspectral Imaging system was used to collect NIR hyperspectral (NIR-H) images. The images were collected from 900-2500 nm with SWIR hyperspectral camera (MRC-303-005-02, Middleton Spectral Vision, Middleton, WI). The spectral camera resolution was 384×288 pixels and pixel size were 24×24 μm. Data were collected using the following parameters: transmission mode, integration time 7.020 ms, frame rate 75 MHz and scan axis speed 0.2 inch/sec. Prior to actual sample measurements, a white reference image and a dark reference image were obtained. The data acquisition software used was Middleton Spectral Vision (Middleton Spectral Vision, Middleton, WI) and data analysis software was Prediktera Evince™ (Prediktera AB, Umea, Sweden).

### X-Ray Powder Diffraction (XRPD)

Bruker D2 Phaser SSD 160 diffractometer (Bruker AXS, Madison, WI) was employed to collect spectra on the samples. The instrument was equipped with the LYNXEYE scintillation detector and Cu Kα radiation (λ=1.54184 Å) at a voltage of 30 KV and a current of 10 mA. Approximately 500 mg of a sample were placed in a sample holder and scanned over 2θ range of 5-35° with a step size of 0.020209° at 1s per step (1241 total steps). Average diffractogram was obtained by rotating samples at 15 rpm during the measurement. Diffract.Suite™ V4.2.1 (Bruker AXS, Madison, WI) and Diffract.Eva V4.2.1 (Bruker AXS, Madison, WI) software was used for data collection and analysis, respectively.

### Poorly Water-Soluble Drugs (PSDs) Studied

The physicochemical properties of the PSDs used in the following examples are reported in Table 2.

TABLE 2
Drug physicochemical properties.

| | | | Number of | Number of | | | Melting |
|---|---|---|---|---|---|---|---|
| Drug | MW | | Hydrogen | Hydrogen | BCS | Exp- | Point |
| (Abbreviation) | (g/mol) | pKa | Donor | Acceptor | Class | LogP | (° C.) |
| Rifaximin | 785.9 | 7 | 5 | 12 | 4 | 2.6 | 228 |
| (RFX) | | | | | | | |
| Dolutegravir | 419.4 | 8 | 2 | 8 | 2 | 2.2 | 193 |
| (DLT) | | | | | | | |
| Cyclosporin | 1202.6 | — | 5 | 12 | 4 | 2.9 | 151 |
| (CYS) | | | | | | | |
| Sirolimus | 914.2 | 10 | 3 | 13 | 2 | 4.3 | 194 |
| (SRL) | | | | | | | |
| Aprepitant | 534.4 | 10 | 2 | 12 | 4 | 4.8 | 255 |
| (APT) | | | | | | | |
| Carbamazepine | 236.3 | 14 | 1 | 1 | 2 | 2.9 | 192 |
| (CBZ) | | | | | | | |
| Aripiprazole | 488.4 | 8 | 2 | 4 | 4 | 4.6 | 139 |
| (APZ) | | | | | | | |
| Tacrolimus | 804.0 | 10 | 3 | 12 | 2 | 3.3 | 133 |
| (TAC) | | | | | | | |
| Dasatinib | 488.0 | 3.1, 6.8 | 4 | 10 | 2 | 2.7 | 286 |
| (DST) | | and 10.8 | | | | | |
| Itraconazole | 705.6 | 3.7 | 0 | 9 | 2 | 5.7 | 170 |
| (ITZ) | | | | | | | |

### Example 1

### Solubility Study

SAIB was heated to 150° C. (RFX, DST, DLT, CYS, ITZ, SRL, APT and CBZ) or 130° C. (TAC and APZ), followed by addition of a known amount of the drug to dissolve in 30 min. Approximately, 100 mg of each sample were transferred to a 100-mL volumetric flask, and volume was made up with the mobile phase to dissolve the drug. The amount of dissolved drug was determined by validated HPLC. The results are shown in Table 3.

TABLE 3
Solubility of PSD in SAIB.

| Poorly | SAIB | Solubility | Increase in |
|---|---|---|---|
| Water-Soluble | Temperature | in SAIB | Solubility Relative |
| Drug | (° C.) | (mg/g) | to Water* |
| Rifaximin | 150 | 36.4 ± 0.9 | 5121.1x |
| Dolutegravir | 150 | 1.7 ± 0.4 | 18.4x |
| Cyclosporin | 150 | 239.0 ± 12.6 | 47800.0x |
| Sirolimus | 150 | 1.9 ± 0.0 | 738.5x |
| Aprepitant | 150 | 0.4 ± 0.0 | 55.7x |
| Carbamazepine | 150 | 76.5 ± 4.0 | 532.3x |
| Aripiprazole | 130 | 21.6 ± 2.6 | 10800.0x |
| Tracolimus | 130 | 115.1 ± 2.3 | 9591.7x |
| Dasatinib | n/a | insoluble | n/a |
| Itraconazole | n/a | insoluble | n/a |

*Water solubility measured at 25 or 37° C. and atmospheric pressure.

### Observations of Solubility Data

The physicochemical properties of the drugs had an influence over their solubility in SAIB. The molecular weight/mass of a drug showed a positive effect on its solubility. For example, CYS, TAC, and RFX showed solubilities of 239.0±12.6, 115.1±2.3, and 36.4±0.9 mg/g, respectively. The molecular weights of CYS, TAC, and RFX are 1202.6, 804, and 785.9 g/mol, respectively. A positive correlation coefficient of 0.60 was obtained between drug solubility in SAIB and their molecular weight.

The number of donor hydrogen (total number of nitrogen-hydrogen and oxygen-hydrogen bonds) had a positive effect on the solubility of the drug in SAIB. In general, the higher the total number of donor hydrogen, the higher the solubility in SAIB. For example, CYS and TAC have five and three donor hydrogens, respectively, and their corresponding solubilities were 239.0±12.6 and 115.1±2.3 mg/g. The correlation coefficient value between hydrogen donor and solubility was 0.52.

On the other hand, the partition coefficient and the melting point of the drugs showed an inverse relationship with the drugs' solubility in SAIB. The higher the melting of a drug, the lower its solubility in the SAIB tended to be, when solubilized at the same temperature. For example, CYS, CBZ, and APT have melting point ranges of 148-151, 189-192, and 251-255° C., respectively, and their corresponding solubilities were 239.0±12.6, 76.5±4.0, and 0.4±0.0 mg/g, respectively. A higher melting point means higher lattice energy, which translated into higher thermal energy needed to overcome the forces that hold the molecules together. The correlation coefficient was −0.53 between the drugs' melting point and their solubility in the SAIB.

Similarly, the partition coefficient (log P) of a drug had to be optimum to solubilize in the SAIB. A too high or too low log P tended to have a negative impact on solubility. The log P of the studied drugs varied from 2.2 to 5.7. The drugs showing high solubility were RFX, CYS, TAC, and CBZ; the log P of these drugs varied from 2.6 to 3.3. On the other hand, the drugs having a log P below 2.6 or above 3.3 showed very little or negligible solubility. It is surprising that drugs with a high log P exhibited low solubility in SAIB even though the log P of SAIB is 6. Nevertheless, negative correlation coefficient of −0.34 was obtained between the drugs' log P and their solubility in the SAIB.

### Example 2

### Stability Study

Physical mixtures and amorphous solid solutions (AmSSols) of various PSDs were prepared by a solubilization-heating method.

The physical mixtures were prepared by heating about 1 g of SAIB to 50° C., the heating was turned off, then about 1 g of the PSD was added to the heated SAIB. The mixture was stirred with a spatula as it cooled to room temperature.

The AmSSols were prepared by heating about 5 g of SAIB to 130 or 150° C., followed by addition of the PSD at 130 or 150° C. with stirring until no crystal was visually observed. The amount of each PSD added is reported in Table 4.

TABLE 4
Amount of PSD in AmSSol.

| Poorly Water- | SAIB Temperature | Amount Added |
|---|---|---|
| Soluble Drug | (° C.) | (mg) |
| Rifaximin | 150 | 181.8 |
| Dolutegravir | 150 | 8.5 |
| Cyclosporin | 150 | 1195 |
| Sirolimus | 150 | 9.6 |
| Aprepitant | 150 | 1.95 |
| Carbamazepine | 150 | 382.7 |
| Aripiprazole | 130 | 108 |
| Tracolimus | 130 | 575.5 |

The prepared AmSSol samples were placed in uncapped scintillation vials and exposed to 40° C./75% RH (Model D-78502, Binder Inc, Bohemia, NY) for one week. The AmSSols were exposed to the elevated temperature and humidity to assess the phase transformation of the dissolved drug. The samples were visually examined and characterized by XRPD, NIR, and NIR-H.

### Visual Results

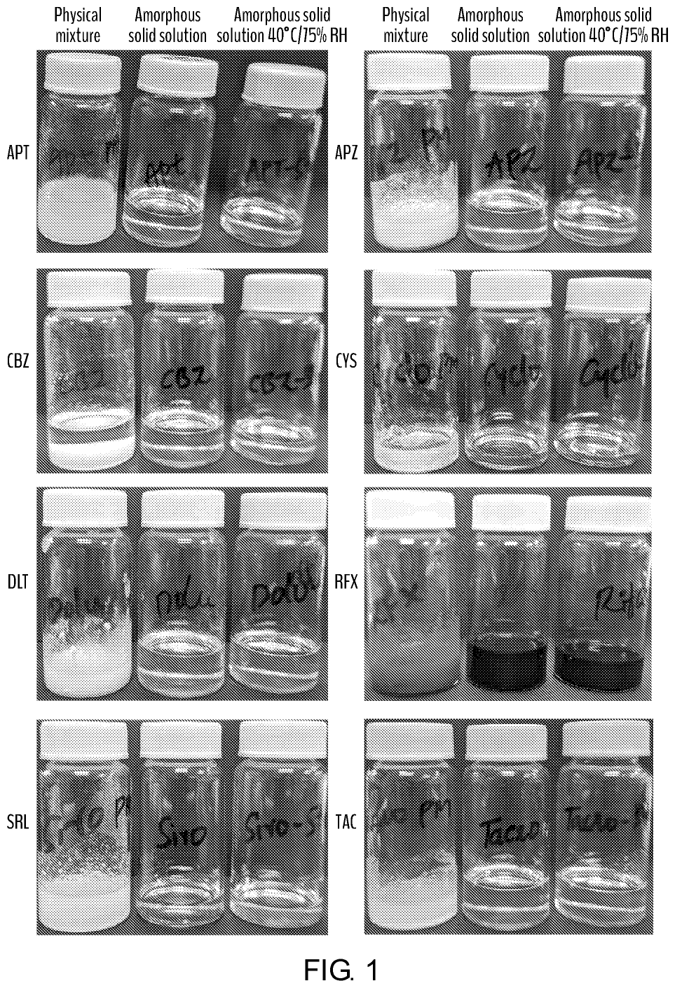

For each PSD, FIG. 1 shows photographs of the physical mixture immediately after preparation and of the AmSSol immediately after preparation as well as after exposure to 40° C./75% RH for one week. As seen in FIG. 1, the physical mixtures were cloudy and/or contained visible crystals. On the other hand, the AmSSols were all clear and colorless or yellowish glassy solids, except for the one containing RFX, which was a dark red glassy solid. The color acquired by the solution depended upon the color of the drug. After one week of exposure, no crystallization of the molecularly dissolved drug was observed in the AmSSols.

### NIR Results

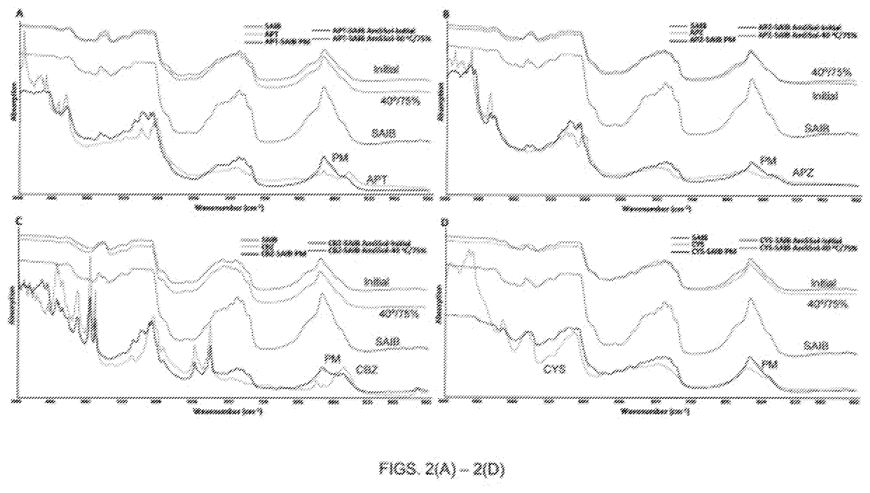

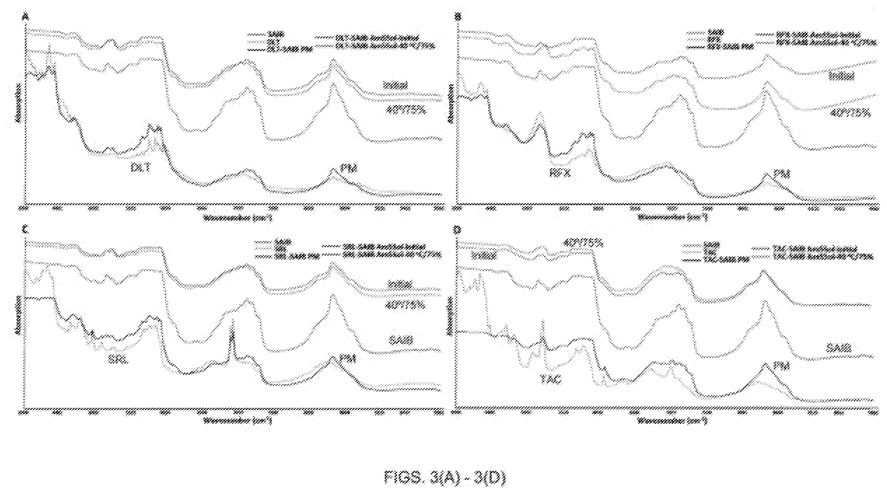

Unlike infrared spectrum, bands in the near infrared spectrum are broad and overlapping. This is due to bond vibration (like O—H, C—H, and N—H) due to overtones and combinations process. However, some of the peaks are sharp, e.g., nitrile group, N—H bending, methyl, methylene, and methine groups, etc. This technique is widely used for both qualitative and quantitative analysis in the pharmaceutical industry. NIR can also be used to characterize amorphous and crystalline forms of the drug. The NIR spectra of the various samples tested are shown in FIGS. 2 and 3.

In particular, FIG. 2(A) shows the NIR spectra of the SAIB alone, the aprepitant (APT) alone, the APT-SAIB physical mixture immediately after preparation, and the APT-SAIB AmSSol before and after exposure to 40° C./75% RH for one week.

FIG. 2(B) shows the NIR spectra of the SAIB alone, the aripiprazole (APZ) alone, the APZ-SAIB physical mixture immediately after preparation, and the APZ-SAIB AmSSol before and after exposure to 40° C./75% RH for one week.

FIG. 2(C) shows the NIR spectra of the SAIB alone, the carbamazepine (CBZ) alone, the CBZ-SAIB physical mixture immediately after preparation, and the CBZ-SAIB AmSSol before and after exposure to 40° C./75% RH for one week.

FIG. 2(D) shows the NIR spectra of the SAIB alone, the cyclosporin (CYS) alone, the CYS-SAIB physical mixture immediately after preparation, and the CYS-SAIB AmSSol before and after exposure to 40° C./75% RH for one week.

FIG. 3(A) shows the NIR spectra of the SAIB alone, the dolutegravir (DLT) alone, the DLT-SAIB physical mixture immediately after preparation, and the DLT-SAIB AmSSol before and after exposure to 40° C./75% RH for one week.

FIG. 3(B) shows the NIR spectra of the SAIB alone, the rifaximin (RFX) alone, the RFX-SAIB physical mixture immediately after preparation, and the RFX-SAIB AmSSol before and after exposure to 40° C./75% RH for one week.

FIG. 3(C) shows the NIR spectra of the SAIB alone, the sirolimus (SRL) alone, the SRL-SAIB physical mixture immediately after preparation, and the SRL-SAIB AmSSol before and after exposure to 40° C./75% RH for one week.

FIG. 3(D) shows the NIR spectra of the SAIB alone, the tacrolimus (TAC) alone, the TAC-SAIB physical mixture immediately after preparation, and the TAC-SAIB AmSSol before and after exposure to 40° C./75% RH for one week.

As seen from FIGS. 2 and 3, the SAIB alone and the drug alone showed distinct spectra bands due to overtone and combinations vibration of the bonds. The drugs showed characteristic peaks in the region 4000-6500 cm⁻¹ due to C—H, O—H, and/or N—H starching vibrations of functional groups present in the molecules. The physical mixtures of SAIB and the drug showed additive spectra encompassing characteristic peaks of the individual components. Furthermore, the intensities of the peaks had reduced due to dilution with the SAIB. In the AmSSols, on the other hand, the characteristic peaks of the drug disappeared completely, which is believed to be attributable to solubilization in the SAIB.

### NIR-H Results

NIR-H is a spectral imaging technique which provides spectral and spatial information about the samples. In pharmaceutical analysis, it is used for qualitative and quantitative analysis. NIR-H data need chemometric methods to construct the images. Most used methods include partial least square regression (PLSR) and principal component analysis (PCA). PCA is used for qualitative purpose, while PLSR is for quantitative analysis. Examples of qualitative analysis include grouping of similar types of samples, distribution of components in a mixture, homogeneity, etc.

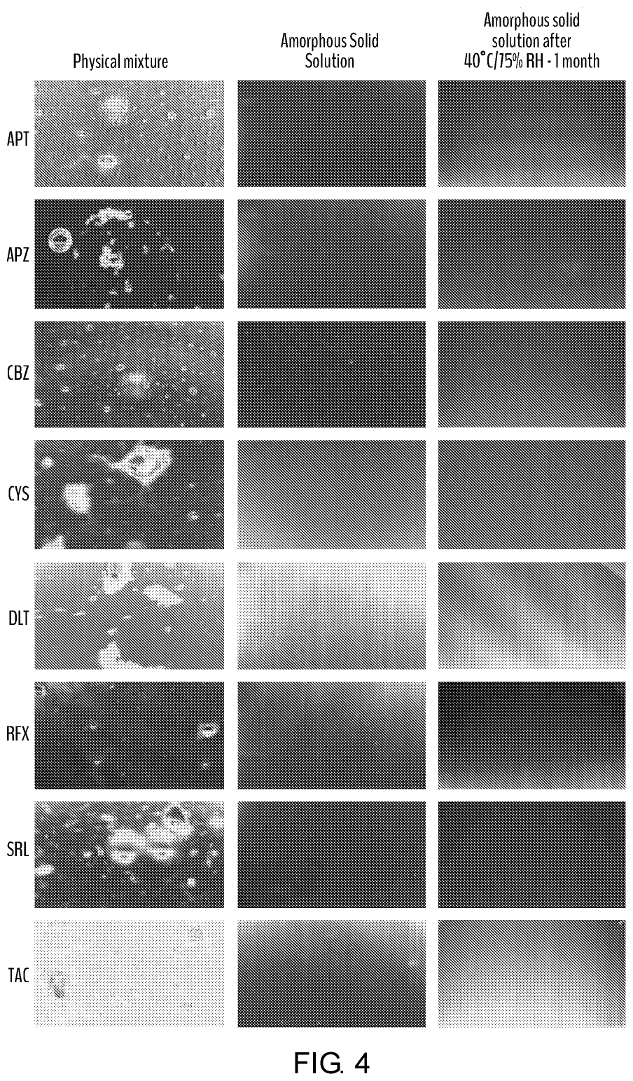

PCA was applied to NIR-H data to create the images in this example. The data were mean centered and treated with standard normal variate. The PCA images of the physical mixtures and the AmSSols in this example are shown in FIG. 4.

In particular, for each PSD tested, FIG. 4 shows the NIR hyperspectroscopy image of the physical mixture immediately after preparation and of the AmSSol immediately after preparation as well as after exposure to 40° C./75% RH for one week.

As seen in FIG. 4, the images of the physical mixtures showed two distinct colored pixels, from yellow to red and blue (or in black and white—dark or light-colored spots). Furthermore, the distribution of the pixels was not uniform, and the images showed the region rich in yellow to red and blue pixels (or dark or light-colored spots), which indicated that two or more components were present. These images confirm that the components were physically mixed and not present at the molecular level. On the other hand, the images of the AmSSols showed only one uniform colored pixel (or in black and white—shades of black or dark or light gray), indicating either only one component was present, or the components were mixed at the molecular level.

### XRPD Results

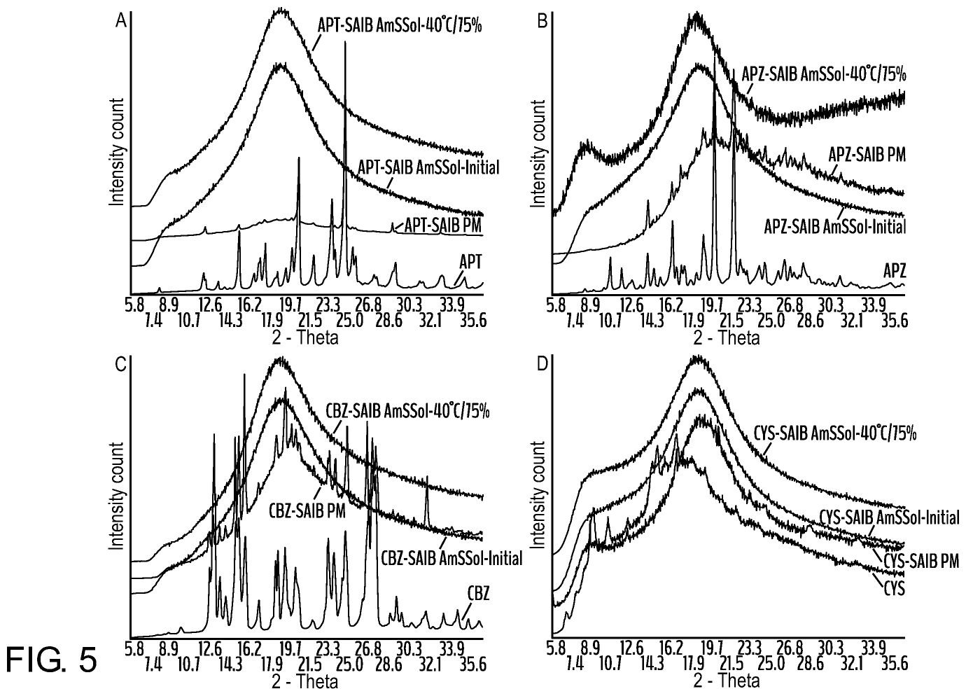

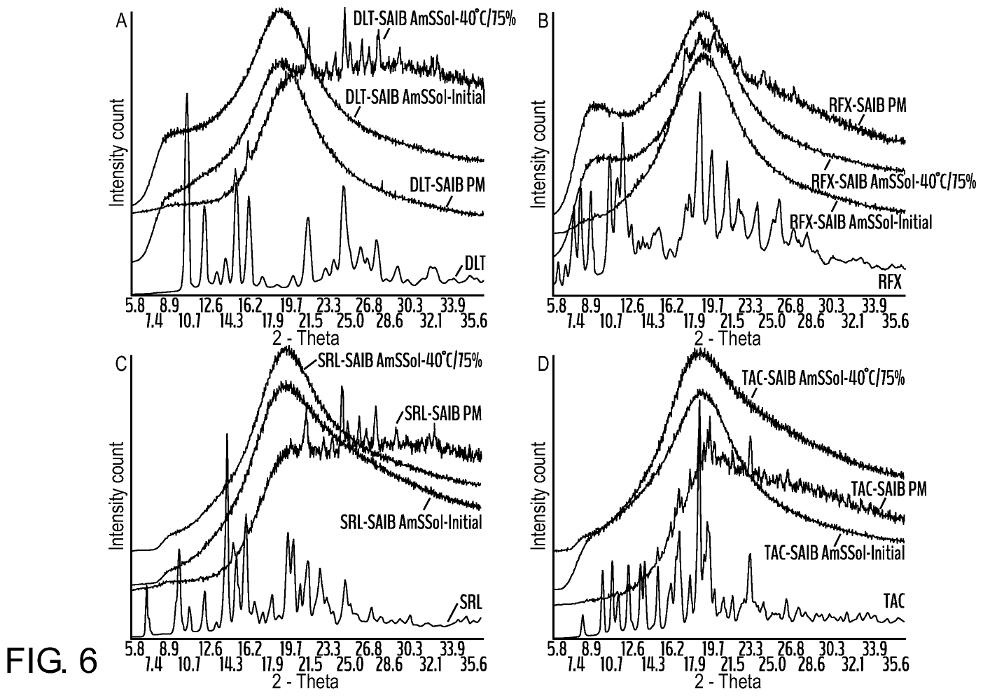

The XRPD data are shown in FIGS. 5 and 6.

In particular, FIG. 5(A) shows the X-ray powder diffractograms of aprepitant (APT) alone, the APT-SAIB physical mixture immediately after preparation, and the APT-SAIB AmSSol before and after exposure to 40° C./75% RH for one week.

FIG. 5(B) shows the X-ray powder diffractograms of aripiprazole (APZ) alone, the APZ-SAIB physical mixture immediately after preparation, and the APZ-SAIB AmSSol before and after exposure to 40° C./75% RH for one week.

FIG. 5(C) shows the X-ray powder diffractograms of carbamazepine (CBZ) alone, the CBZ-SAIB physical mixture immediately after preparation, and the CBZ-SAIB AmSSol before and after exposure to 40° C./75% RH for one week.

FIG. 5(D) shows the X-ray powder diffractograms of the cyclosporin (CYS) alone, the CYS-SAIB physical mixture immediately after preparation, and the CYS-SAIB AmSSol before and after exposure to 40° C./75% RH for one week.

FIG. 6(A) shows the X-ray powder diffractograms of dolutegravir (DLT) alone, the DLT-SAIB physical mixture immediately after preparation, and the DLT-SAIB AmSSol before and after exposure to 40° C./75% RH for one week.

FIG. 6(B) shows the X-ray powder diffractograms of rifaximin (RFX) alone, the RFX-SAIB physical mixture immediately after preparation, and the RFX-SAIB AmSSol before and after exposure to 40° C./75% RH for one week.

FIG. 6(C) shows the X-ray powder diffractograms of sirolimus (SRL) alone, the SRL-SAIB physical mixture immediately after preparation, and the SRL-SAIB AmSSol before and after exposure to 40° C./75% RH for one week.

FIG. 6(D) shows the X-ray powder diffractograms of tacrolimus (TAC) alone, the TAC-SAIB physical mixture immediately after preparation, and the TAC-SAIB AmSSol before and after exposure to 40° C./75% RH for one week.

As seen in FIGS. 5 and 6, each drug showed its high-intensity characteristic peaks at 2-theta as follows: APT (15.1, 17.5, 20.4, 21.8, 24.6, and 29.0°); APZ (13.4, 16.2, 18.9, 20.0, and 21.7°); CBZ (12.9, 14.9, 15.1, 15.7, 18.4, 18.6, 19.2, 20.2, 23.1, 23.6, 24.7, 26.7, and 27.1°); CYS (9.1, 10.4, 14.9, and 16.5°); DLT (10.4, 12.0, 14.8, 15.9, 21.2, and 24.3°); RFX (7.3, 7.9, 8.8, 10.5, 11.7, 18.6, 19.6, and 21.0°); SRL (6.9, 9.8, 12.1, 14.1, 14.9, 15.8, 19.5, 20.0, and 21.3°); and TAC (9.9, 10.7, 12.2, 13.3, 13.7, 14.9, 16.8, 18.6, 19.3, and 23.1°). The physical mixtures of SAIB and the drugs showed the characteristic peaks of the drugs, except in SRL. This was probably due to polymorphic transformation in the presence of SAIB. In the AmSSols, the crystalline peaks of the drugs completely disappeared. This can be explained by solubilization of the drugs in the SAIB. Thus, the SAIB acted as a solvent for the drugs, resulting in the drug solutions.

### Observations of Stability Data

Amorphous drug is thermodynamically instable and devitrify when exposed to exaggerated conditions, such as high temperature and humidity. These conditions increase molecular mobility by lowering the glass transition temperature. The prepared AmSSols were exposed to 40° C./75% RH to accelerate devitrification, if any. After exposure to the elevated temperature and humidity conditions, there was no sign of drug crystals in the AmSSols (FIG. 1). The NIR-H images showed no significant changes in the pixels to indicate drug crystallization (FIG. 4). Similarly, NIR and XRPD showed no significant changes in the spectra and diffractograms after the exposure (FIGS. 2, 3, 5, and 6). The NIR spectra of the initial samples almost overlapped with the exposed samples. If there was crystallization, the spectra for the AmSSols should look like those for the physical mixtures. Similarly, the XRPD diffractograms were halo characteristics of amorphous or glassy materials. Without wishing to be bound by theory, the stability of the drugs against crystallization in the AmSSols based on the SAIB could be explained by several phenomena occurring simultaneously, such as the hydrophobicity of the SAIB, the hydrophobic interaction between drug and SAIB molecules, the glassy nature of the SAIB, and the entrapping of the drug in the glassy matrix, which minimizes drug interaction with the aqueous phase during exposure to the elevated conditions.

### Example 3

### Preparation of Amorphous Solid Dispersion Formulations

Drug (aprepitant) and various amounts of SAIB were dissolved in the least quantity of ethanol.

Hydrophilic excipient or excipients (lactose, microcrystalline cellulose, croscarmellose sodium, and/or sodium lauryl sulfate, etc.) were sieved through a #18 mesh screen and blended in a V-blender (V-blender, Model VH-2) or a high-shear granulator (KG5, KEY International Inc., NJ, USA) for 2 min, followed by granulation with a solution of the drug and the SAIB. The drug/SAIB solution was gradually added to the mixture of excipient(s) with impeller rotation (KG5, KEY International Inc., NJ, USA).

The ethanol was removed from the granules by drying in an oven at 50° C. until loss on drying was less than 1%.

The dried granules were milled (Quadro Comil®, Model-193, Quadro Engineering Inc., Waterloo, Canada) and sieved through a #18 mesh screen.

The milled granules were further dried in a vacuum oven for 24 hours.

A super-disintegrant mixture (sodium croscarmellose/sodium starch glycolate/Kollidon) was added to the dried granules, followed by lubrication with magnesium stearate/colloidal silicon dioxide/talc for 2 min in a V-blender.

The final blend was compressed into tablets using a Mini Press-1 (Globe Pharma, New Brunswick, NJ, USA) 10-station tableting machine with 8/10 mm biconvex and punches (Natoli Engineering Company, Saint Charles, MO, USA).

The final blend was also filled into hard gelatin capsules.

The compositions of the prepared tablets/capsule are reported in Table 5.

TABLE 5
Compositions of aprepitant AmSD based on SAIB and MCC.

| | Quantity in mg | | | |
|---|---|---|---|---|
| Component | F1 | F2 | F3 | F4 |
| Aprepitant | 40 | 40 | 40 | 40 |
| SAIB | 20 | 40 | 60 | 80 |
| Microcrystalline | 120 | 200 | 200 | 200 |
| cellulose | | | | |
| Croscarmellose | 36 | 35.6 | 37.8 | 40 |
| sodium | | | | |
| Tween 80 | 36 | — | — | — |
| Sodium lauryl sulfate | — | 40 | 40 | 40 |
| Dosage form | Capsule | Tablet | Tablet | Tablet |

### Example 4

### Dissolution Testing

Each of the tablets and capsule from Example 3 was added to 900 ml of a 0.4% aqueous solution of sodium lauryl sulfate at a paddle speed of 75 rpm and a temperature of 37° C. for 2 hours to determine their dissolution rate.

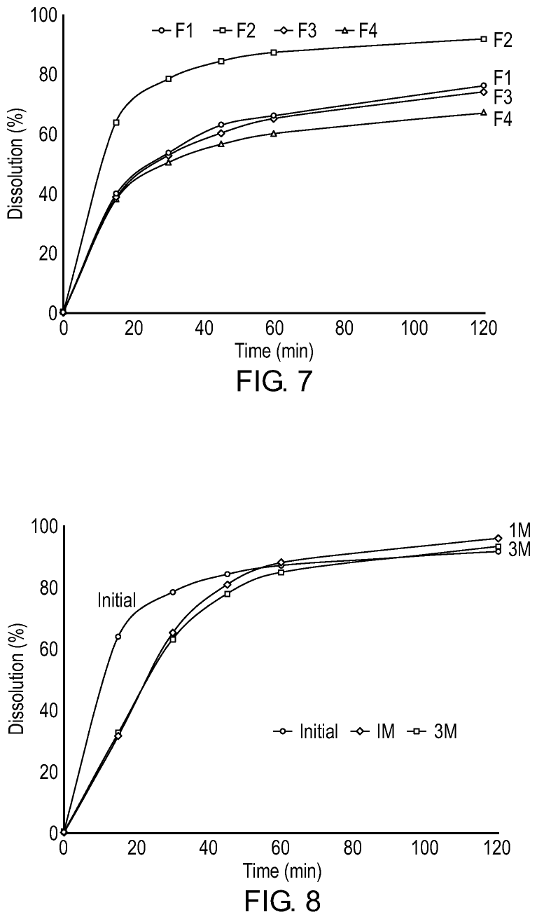

The dissolution rate of each formulation, F1-F4, is shown in FIG. 7.

As seen in FIG. 7, dissolution varied from 48.0-91.9% in 2 hours as a function of the aprepitant-to-SAIB mass ratio. Dissolution decreased with an increase in the SAIB concentration in the formulation. For example, dissolution decreased from 91.9 (F2) to 67.1% (F4) by increasing the aprepitant:SAIB mass ratio from 1:1 (F1) to 1:2 (F2).

### Example 5

### Dissolution Stability Testing

The formulation F2 from Example 3 was aged at 40° C./75% RH for 1 and 3 months. The dissolution rates of the initial, 1-month-old, and 3-month-old tablets were measured according to the method described in Example 4.

The dissolution rate of each tablet is shown in FIG. 8.

As seen from FIG. 8, there was insignificant change in the extent of dissolution in the aged samples compared to the initial sample. For example, the dissolution was 91.9 and 93.7% in 2 hours before and after exposure to 40° C./75% RH for three months, respectively.

### Example 6

### Dissolution Stability Testing

The formulation F2 from Example 3 was aged at 25° C./60% RH for 3 months. The dissolution rates of the initial and 3-month-old tablets were measured according to the method described in Example 4.

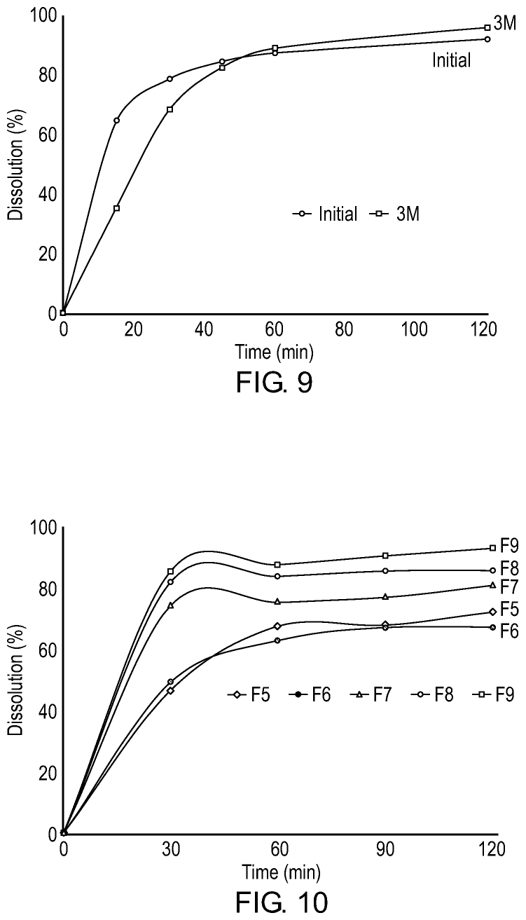

The dissolution rate of each tablet is shown in FIG. 9.

As seen in FIG. 9, no significant change in the extent of dissolution was observed after storage at 25° C./60% RH for three months. The dissolution value was 91.9 and 95.7% in 2 hours before and after exposure to 25° C./60% RH for three months, respectively.

### Example 7

### Bioavailability Testing

The formulation F2 from Example 3 was further assessed for oral bioavailability in beagle dogs (n=4/formulation) and compared against tablets made of a physical mixture of the same drug and excipients, but without the SAIB.

The compositions of F2 and the physical mixture are reported in Table 6.

TABLE 6
Tablet composition of aprepitant AmSD and physical mixture.

Quantity in mg

| | Amorphous | |
|---|---|---|
| | Solid | Physical |
| Component | Dispersion | Mixture |
| Aprepitant | 40 | 40 |
| SAIB | 40 | — |
| Microcrystalline cellulose | 200 | 200 |
| Sodium lauryl sulfate | 40 | 40 |
| Croscarmellose sodium | 35.6 | 35.6 |

The bioavailability data for the tablets of F2 and the physical mixture are reported in Table 7.

TABLE 7
Pharmacokinetic parameters of aprepitant
AmSD and physical mixture.

| Pharmacokinetic | Amorphous solid | |
|---|---|---|
| parameters | dispersion | Physical mixture |
| Cₘₐₓ (μg/ml) | 2.5 ± 0.4 | 1.2 ± 0.2 |
| AUC (ng/ml · h) | 25.7 ± 2.7 | 9.8 ± 0.7 |

As seen in Table 7, there was a significant increase in the bioavailability parameters when aprepitant was administered as an AmSD formulation using SAIB as a carrier compared to a physical mixture without the SAIB. The Cₘₐₓ and AUC of the AmSD formulation were 2.1 and 2.6-fold those of the physical mixture, respectively.

### Example 8

### Preparation of Amorphous Solid Dispersion Formulations

Tablets and capsules were prepared using the procedures outline in Example 3 with tacrolimus as the PSD.

The compositions of the prepared tablet/capsules are reported in Table 8.

TABLE 8
Compositions of tacrolimus AmSD based on SAIB and MCC.

| | Quantity in mg | | | | |
|---|---|---|---|---|---|
| Component | F5 | F6 | F7 | F8 | F9 |
| Tacrolimus | 5 | 5 | 5 | 5 | 5 |
| SAIB | 7.5 | 10 | 7.5 | 7.5 | 7.5 |
| Microcrystalline | 50 | 50 | 50 | 50 | 50 |
| cellulose | | | | | |
| Croscarmellose | 10 | 10 | 10 | 15 | 15 |
| sodium | | | | | |
| Sodium lauryl | — | — | 5 | 5 | 5 |
| sulfate | | | | | |
| Dosage form | Capsule | Capsule | Capsule | Capsule | Tablet |

### Example 9

### Dissolution Testing

Each of the capsules and tablet from Example 8 was added to 900 ml of a 0.4% aqueous solution of sodium lauryl sulfate at a paddle speed of 75 rpm and a temperature of 37° C. for 2 hours to determine their dissolution rate.

The dissolution rate of each formulation, F5-F9, is shown in FIG. 10.

As seen in FIG. 10, the dissolution rate and extent were affected by the composition of the formulation, especially SAIB and excipients. Dissolution was less when the mass ratio of tacrolimus-to-SAIB was 1:2 compared to when the ratio was 1:1.5. For example, the dissolution was 72.8 and 67.7% in 2 hours for the F5 and F6 formulations, respectively. Dissolution was increased by an increase in the amount and/or percentage of excipients. For example, the dissolution was 72.8% for F5, and 86.3% for F8 when the croscarmellose sodium and sodium lauryl sulfate concentrations increased. Furthermore, the dissolution of the tablet was higher than the dissolution of the capsules. For example, the dissolution was 86.3 and 93.5% in 2 hours for F8 (capsule) and F9 (tablet), respectively.

### Example 10

### Dissolution Stability Testing

The formulation F9 from Example 8 was aged at 40° C./75% RH for 1 and 2 months. The dissolution rates of the initial, 1-month-old, and 2-month-old tablets were measured according to the method described in Example 9.

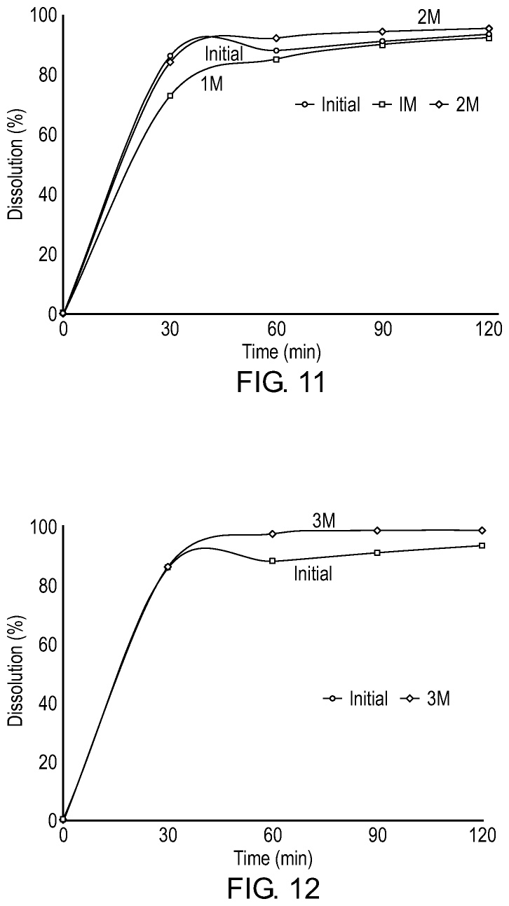

The dissolution rate of each tablet is shown in FIG. 11.

As seen in FIG. 11, insignificant differences in the rate and extent of dissolution were observed between the initial and the exposed formulations. Dissolution was 93.5 and 95.6% before and after exposure to 40° C./75% RH, respectively; thus, indicating formulation stability at accelerated conditions.

### Example 11

### Dissolution Stability Testing

The formulation F9 from Example 8 was aged at 25° C./60% RH for 3 months. The dissolution rates of the initial and 3-month-old tablets were measured according to the method described in Example 9.

The dissolution rate of each tablet is shown in FIG. 12.

As seen in FIG. 12, the aged formulation was stable against crystallization as indicated by no significant change in dissolution. Dissolution was 93.5 and 98.7% initially and after exposure at 25° C./60% RH for 3 months, respectively.

### Example 12

### Bioavailability Testing

The formulation F9 from Example 8 was further assessed for oral bioavailability in beagle dogs (n=4/formulation) and compared against tablets made of a physical mixture of the same drug and excipients, but without the SAIB.

The compositions of F9 and the physical mixture are reported in Table 9.

TABLE 9
Tablet Composition of tacrolimus AmSD and physical mixture.

Quantity in mg

| | Amorphous | |
|---|---|---|
| | Solid | Physical |
| Component | Dispersion | Mixture |
| Tacrolimus | 5 | 40 |
| SAIB | 7.5 | — |
| Microcrystalline cellulose | 50 | 50 |
| Sodium lauryl sulfate | 5 | 5 |
| Croscarmellose sodium | 15 | 15 |

The bioavailability data for the tablets of F9 and the physical mixture are reported in Table 10.

TABLE 10
Pharmacokinetic parameters of tacrolimus
AmSD and physical mixture.

| Pharmacokinetic | Amorphous solid | |
|---|---|---|
| parameters | dispersion | Physical mixture |
| Cₘₐₓ (μg/ml) | 38.0 ± 6.1 | 20.6 ± 3.6 |
| AUC (ng/ml · h) | 248.7 ± 196.5 | 129.9 ± 31.1 |

As seen in Table 10, the bioavailability of tacrolimus increased significantly as indicated by a significant increase in the pharmacokinetic parameters. The Cₘₐₓ and AUC of the AmSD formulation of tacrolimus was 1.8 and 1.9-fold of those of the physical mixture, respectively.

### EXEMPLARY EMBODIMENTS

Exemplary embodiments include the following:
1. A pharmaceutical composition comprising:
   (a) a poorly water-soluble drug (PSD);
   (b) sucrose acetate isobutyrate (SAIB);
   (c) less than 0.01 wt % of a solvent, based on the total weight of the composition; and
   (d) optionally, at least one pharmaceutically acceptable excipient which is insoluble in the SAIB,
   wherein the composition:
      (i) is an amorphous solid solution or an amorphous solid dispersion;
      (ii) has a mass ratio of PSD to SAIB ranging from 0.1:1 to 1:5; and
      (iii) has a mass ratio of excipient to SAIB ranging from 0:1 to 100:1, and wherein the PSD is present in the molecular or amorphous state in the SAIB.
2. The pharmaceutical composition according to embodiment 1, which does not comprise the at least one excipient and which is an amorphous solid solution.
3. The pharmaceutical composition according to embodiment 2, which has an extended-release profile.
4. The pharmaceutical composition according to embodiment 1, which comprises the at least one excipient and which is an amorphous solid dispersion.
5. The pharmaceutical composition according to embodiment 4, which has a mass ratio of excipient to SAIB ranging from 5:1 to 100:1 and which has an immediate-release profile.
6. The pharmaceutical composition according to embodiment 4, which has a mass ratio of excipient to SAIB ranging from 0.01:1 to 4:1 or from 0.01:1 to 1:1, and which has an extended-release profile.
7. The pharmaceutical composition according to any one of the preceding embodiments, wherein the PSD has a solubility in the SAIB of at least 0.1 mg/g, at least 0.4 mg/g, at least 1.0 mg/g, at least 1.5 mg/g, at least 2.0 mg/g, at least 10 mg/g, at least 20 mg/g, at least 30 mg/g, at least 50 mg/g, at least 75 mg/g, at least 100 mg/g, at least 150 mg/g, or at least 200 mg/g, at 130° C. or 150° C. and atmospheric pressure.
8. The pharmaceutical composition according to any one of the preceding embodiments, wherein the PSD has a molecular mass of at least 500 g/mol, at least 750 g/mol, at least 1000 g/mol, or at least 1200 g/mol.
9. The pharmaceutical composition according to any one of the preceding embodiments, wherein the PSD has a total number of donor hydrogen of at least 1, at least 2, at least 3, at least 4, or at least 5 per molecule.
10. The pharmaceutical composition according to any one of the preceding embodiments, wherein the PSD has a melting point of 250° C. or less, 225° C. or less, 200° C. or less, 195° C. or less, 190° C. or less, 185° C. or less, 180° C. or less, 175° C. or less, 170° C. or less, 165° C. or less, 160° C. or less, 155° C. or less, or 150° C. or less.
11. The pharmaceutical composition according to any one of the preceding embodiments, wherein the PSD has a partition coefficient (log P) of 2.6 to 3.3.
12. The pharmaceutical composition according to any one of the preceding embodiments, wherein the PSD comprises aprepitant, aripiprazole, carbamazepine, cyclosporine, dolutegravir, rifaximin, sirolimus, or tacrolimus.
13. The pharmaceutical composition according to any one of the preceding embodiments, which comprises 0.001 wt % or less of solvent.
14. The pharmaceutical composition according to any one of the preceding embodiments, which is free of solvent.
15. The pharmaceutical composition according to any one of the preceding embodiments, which is free of cellulose acetate butyrate.
16. The pharmaceutical composition according to any one of the preceding embodiments, which is stable when stored in an open container at 40° C. and 75% relative humidity for at least one week.
17. An oral dosage form comprising the pharmaceutical composition according to any one of the preceding embodiments.
18. The oral dosage form according to embodiment 17, which is a tablet or a capsule.
19. The oral dosage form according to any one of embodiments 17-18, which comprises from 0.5 to 500 mg of the PSD.
20. The oral dosage form according to any one of embodiments 17-19, which has a higher dissolution rate in water than an equivalent oral dosage form without the SAIB.
21. The oral dosage form according to any one of embodiments 17-20, which has a higher serum maximum concentration (Cₘₐₓ) than an equivalent oral dosage form without the SAIB.
22. The oral dosage form according to any one of embodiments 17-21, which has a Cₘₐₓ of at least 1.1×, at least 1.2×, at least 1.3×, at least 1.4×, at least 1.5×, at least 1.6×, at least 1.7×, at least 1.8×, at least 1.9×, or at least 2× greater than the Cₘₐₓ of an equivalent oral dosage form without the SAIB.
23. The oral dosage form according to any one of embodiments 17-22, which has a greater area under the curve (AUC) than an equivalent oral dosage form without the SAIB.
24. The oral dosage form according to any one of embodiments 17-23, which has an AUC of at least at least 1.1×, at least 1.2×, at least 1.3×, at least 1.4×, at least 1.5×, at least 1.6×, at least 1.7×, at least 1.8×, at least 1.9×, at least 2×, at least 2.1×, at least 2.2×, at least 2.3×, at least 2.4×, or at least 2.5× greater than the AUC of an equivalent oral dosage form without the SAIB.
25. A process for preparing the pharmaceutical composition according to any one of embodiments 1-16, the process comprising:
   heating the SAIB to a temperature sufficient to dissolve the PSD and for the PSD to exist in the molecular or amorphous state in the SAIB;
   adding the PSD and optionally, the at least one pharmaceutically acceptable excipient, to the heated SAIB with mixing to dissolve the PSD and to form a mixture; and
   cooling the mixture to form the pharmaceutical composition comprising the amorphous solid solution or the amorphous solid dispersion.
26. The process according to embodiment 25, wherein the SAIB is heated to a temperature from 60 to 200° C., from 70 to 200° C., from 80 to 200° C., from 90 to 200° C., from 100 to 200° C., from 110 to 200° C., from 120 to 200° C., from 130 to 200° C., from 140 to 200° C., from 150 to 200° C., from 80 to 175° C., from 90 to 175° C., from 100 to 175° C., from 110 to 175° C., from 120 to 175° C., from 130 to 175° C., from 140 to 175° C., from 150 to 175° C., from 80 to 160° C., from 90 to 160° C., from 100 to 160° C., from 110 to 160° C., from 120 to 160° C., from 130 to 160° C., from 140 to 160° C., from 150 to 160° C., from 80 to 150° C., from 90 to 150° C., from 100 to 150° C., from 110 to 150° C., from 120 to 150° C., from 130 to 150° C., or from 140 to 150° C.
27. The process according to embodiments 25 or 26, which is carried out in the absence of an organic solvent.
28. The process according to any one of embodiments 25-27, which is carried out in the absence of cellulose acetate butyrate.
29. The process according to any one of embodiments 25-28, wherein one or more steps are carried out in a hot-melt extruder.
30. A process for preparing the pharmaceutical composition according to any one of embodiments 1-16, the process comprising:
   forming a mixture comprising the PSD, the SAIB, an organic solvent, and optionally, the at least one pharmaceutically acceptable excipient;
   granulating the mixture to form granules; and
   removing the organic solvent from the granules to form the pharmaceutical composition comprising the amorphous solid solution or the amorphous solid dispersion.
31. The process according to embodiment 30, wherein the removing step comprises heating, vacuum drying, or both.

The invention has been described in detail with particular reference to specific embodiments thereof, but it will be understood that variations and modifications can be made within the spirit and scope of the invention.

1. A pharmaceutical composition comprising:
(a) a poorly water-soluble drug (PSD);
(b) sucrose acetate isobutyrate (SAIB);
(c) less than 0.01 wt % of a solvent, based on the total weight of the composition; and
(d) optionally, at least one pharmaceutically acceptable excipient which is insoluble in the SAIB,
wherein the composition:
(i) is an amorphous solid solution or an amorphous solid dispersion;
(ii) has a mass ratio of PSD to SAIB ranging from 0.1:1 to 1:5; and
(iii) has a mass ratio of excipient to SAIB ranging from 0:1 to 100:1, and wherein the PSD is present in the molecular or amorphous state in the SAIB.

2. The pharmaceutical composition according to claim 1, which does not comprise the at least one excipient and which is an amorphous solid solution.

3. The pharmaceutical composition according to claim 1, which comprises the at least one excipient and which is an amorphous solid dispersion.

4. The pharmaceutical composition according to claim 3, which has a mass ratio of excipient to SAIB ranging from 5:1 to 100:1 and which has an immediate-release profile.

5. The pharmaceutical composition according to claim 1, wherein the PSD has:
(A) a solubility in the SAIB of at least 0.1 mg/g at 130° C. or 150° C. and atmospheric pressure;
(B) a molecular mass of at least 500 g/mol;
(C) a total number of donor hydrogen of at least 1 per molecule;
(D) a melting point of 250° C. or less; and/or
(E) a partition coefficient (log P) of 2.6 to 3.3.

6. The pharmaceutical composition according to claim 1, wherein the PSD comprises aprepitant, aripiprazole, carbamazepine, cyclosporine, dolutegravir, rifaximin, sirolimus, or tacrolimus.

7. The pharmaceutical composition according to claim 1, which:
(A) comprises 0.001 wt % or less of solvent;
(B) is free of cellulose acetate butyrate; and/or
(D) is stable when stored in an open container at 40° C. and 75% relative humidity for at least one week.

8. An oral dosage form comprising the pharmaceutical composition according to claim 1.

9. The oral dosage form according to claim 8, which:
(A) is a tablet or a capsule;
(B) comprises from 0.5 to 500 mg of the PSD;
(C) has a higher dissolution rate in water than an equivalent oral dosage form without the SAIB;
(D) has a higher serum maximum concentration (Cₘₐₓ) than an equivalent oral dosage form without the SAIB;
(E) has a Cₘₐₓ of at least 1.1× greater than the Cₘₐₓ of an equivalent oral dosage form without the SAIB;
(F) has a greater area under the curve (AUC) than an equivalent oral dosage form without the SAIB; and/or
(G) has an AUC of at least at least 1.1× greater than the AUC of an equivalent oral dosage form without the SAIB.

10. A process for preparing the pharmaceutical composition according to claim 1, the process comprising:
heating the SAIB to a temperature sufficient to dissolve the PSD and for the PSD to exist in the molecular or amorphous state in the SAIB;
adding the PSD and optionally, the at least one pharmaceutically acceptable excipient, to the heated SAIB with mixing to dissolve the PSD and to form a mixture; and
cooling the mixture to form the pharmaceutical composition comprising the amorphous solid solution or the amorphous solid dispersion.

11. The process according to claim 10, wherein the SAIB is heated to a temperature from 60 to 200° C.

12. The process according to claim 10, which is carried out in the absence of an organic solvent.

13. The process according to claim 10, wherein one or more steps are carried out in a hot-melt extruder.

14. A process for preparing the pharmaceutical composition according to claim 1, the process comprising:
forming a mixture comprising the PSD, the SAIB, an organic solvent, and optionally, the at least one pharmaceutically acceptable excipient;
granulating the mixture to form granules; and
removing the organic solvent from the granules to form the pharmaceutical composition comprising the amorphous solid solution or the amorphous solid dispersion.

15. The process according to claim 14, wherein the removing step comprises heating, vacuum drying, or both.

16. The pharmaceutical composition according to claim 2, which has an extended-release profile.

17. The pharmaceutical composition according to claim 3, which has a mass ratio of excipient to SAIB ranging from 0.01:1 to 4:1, and which has an extended-release profile.

18. The pharmaceutical composition according to claim 3, which has a mass ratio of excipient to SAIB ranging from 0.01:1 to 1:1, and which has an extended-release profile.

19. The pharmaceutical composition according to claim 1, which is free of solvent.

20. The process according to claim 10, which is carried out in the absence of cellulose acetate butyrate.
